(19) United States
(12) Patent Application Publication
(10) Pub. No.: US 2018/0312623 A1
(43) Pub. Date: Nov. 1, 2018

(54) Polyurethane Elastomer with High Ultimate Elongation

(71) Applicants: Liang Wang, Acworth, GA (US); Viktoria Wang, Acworth, GA (US)

(72) Inventors: Liang Wang, Acworth, GA (US); Viktoria Wang, Acworth, GA (US)

(21) Appl. No.: 15/582,362

(22) Filed: Apr. 28, 2017

(51) Int. Cl. C08G 18/48; C08G 18/24; C08G 18/22; C08G 18/32; C08G 18/78; C07C 275/06

(52) U.S. Cl. CPC C08G 18/4808; C08G 18/244; C08G 18/227; C07C 275/06; C08G 18/3225; C08G 18/7812; C08G 18/222

(57) ABSTRACT

The present invention relates to polyurethane-urea elastomers with exceptional mechanical properties and enhanced elasticity based on molecular structure analysis. The present invention involves the synthesis of a polyurethane-urea elastomer using a combination of commercially available materials: (1) polyols with ultralow unsaturation and narrow polydispersity, (2) diisocyanates with symmetric steric conformation, and (3) bulky diamines between functional groups. The preparation method for the novel polyurethane-urea is disclosed. The subject elastomers have the highest reported elongation-at-break among known polyurethane elastomers.

### STATEMENT REGARDING FEDERALLY SPONSORED RESEARCH OR DEVELOPMENT

This invention was made with government support under grant contract number NNX16CC45P awarded by NASA. The Government has certain rights in the invention.

### US PATENT DOCUMENTS

| U.S. Pat. No. 9,080,010 B2 | Feb. 17, 2011 | Wolf, et al. | |
|---|---|---|---|
| U.S. Pat. No. 8,273,846 | Sep. 25, 2012 | Nefzger, et al. | 528/85 |
| U.S. Pat. No. 7,511,111 | Mar. 31, 2009 | Lawrey, et al. | 528/59 |
| U.S. Pat. No. 7,045,650 | May 16, 2006 | Lawrey, et al | 560/26 |
| U.S. Pat. No. 6,964,626 | Nov. 15, 2005 | Wu, et al | 474/260 |
| U.S. Pat. No. 6,906,163 | Jun. 14, 2005 | Lawrey, et al. | 528/55 |
| U.S. Pat. No. 6,903,179 | Jun. 7, 2005 | Lawrey, et al. | 528/61 |
| U.S. Pat. No. 6,824,703 | Nov. 30, 2004 | Lawrey, et al. | 252/182.25 |
| U.S. Pat. No. 6,737,497 | May 18, 2004 | Lawrey, et al. | 528/61 |
| U.S. Pat. No. 6,720,403 | Apr. 13, 2004 | Houser | 528/85 |
| U.S. Pat. No. 6,713,599 B1 | Mar. 30, 2004 | Hinz et al. | 528/408 |
| U.S. Pat. No. 6,586,566 B1 | Jul. 1, 2003 | Hofmann, et al. | 528/428 |
| U.S. Pat. No. 6,624,281 | Sep. 23, 2003 | Lawrey, et al. | 528/61 |
| U.S. Pat. No. 6,420,445 | Jul. 16, 2002 | Barksby, et al. | 521/159 |
| U.S. Pat. No. 6,291,388 | Sep. 18, 2001 | Hofmann, et al. | 502/154 |
| U.S. Pat. No. 6,204,357 | Mar. 20, 2001 | Ooms, et al. | 528/406 |
| U.S. Pat. No. 5,981,686 | Nov. 9, 1999 | Waldbauer, Jr. | 528/76 |
| U.S. Pat. No. 5,952,261 | Sep. 14, 1999 | Combs | 502/175 |
| U.S. Pat. No. 5,948,875 | Sep. 7, 1999 | Lin, et al. | 528/61 |
| U.S. Pat. No. 5,843,357 | Dec. 1, 1998 | Seneker, et al. | 264/204 |
| U.S. Pat. No. 5,723,563 | Mar. 3, 1998 | Lawrey, et al. | 528/61 |
| U.S. Pat. No. 5,712,216 | Jan. 27, 1998 | Le-Khac, et al. | 502/175 |
| U.S. Pat. No. 5,708,118 | Jan. 13, 1998 | Seneker, et al. | 528/61 |
| U.S. Pat. No. 5,696,221 | Dec. 9, 1997 | Barksby, et al. | 528/64 |
| U.S. Pat. No. 5,691,441 | Nov. 25, 1997 | Seneker, et al. | 528/61 |
| U.S. Pat. No. 5,648,447 | Jul. 15, 1997 | Seneker, et al. | 528/63 |
| U.S. Pat. No. 5,482,908 | Jan. 9, 1996 | Le-Khac | 502/156 |
| U.S. Pat. No. 5,470,813 | Nov. 23, 1995 | Le-Khac | 502/175 |
| U.S. Pat. No. 5,340,902 | Aug. 23, 1994 | Smith, et al. | 528/61 |
| U.S. Pat. No. 5,158,922 | Oct. 27, 1992 | Hinney, et al. | 502/175 |
| U.S. Pat. No. 5,061,426 | Oct. 29, 1991 | Frauendorf, et al. | 262/205 |
| U.S. Pat. No. 5,000,899 | Mar. 19, 1991 | Dreibelbeis, et al | 264/205 |
| U.S. Pat. No. 4,973,647 | Nov. 27, 1990 | Bretches, et al. | 528/61 |
| U.S. Pat. No. 4,477,589 | Oct. 16, 1984 | van der Hulst, et al. | 502/169 |
| U.S. Pat. No. 4,472,560 | Sep. 18, 1984 | Kuyper, et al. | 526/120 |

### OTHER REFERENCES

Balkazara, J., 2011, “Elastomers: Types, Properties, and Applications (Material Science and Technology,” Primer
Clemitson, I. R., 2015, “Castable Polyurethane Elastomers, Second Edition,” CRC Press
Drobny, J. G., 2013, “Handbook of Thermoplastic Elastomers (Plastics Design Library),” Elsevier
Goff, J., Sulaiman, S., Arbles, B., and Lewicki, J. P., 2016, “Soft Materials with Recoverable Shape Factors from Extreme Distortion States,” Adv. Mater. 26, 2393-2398
Hadjichrichristidis, N., Pispas, S., and Flloudas, G., 2003, “Block Copolymers: Synthetic Strategies, Physical Properties, and Applications,” John Wiley and Sons, Inc.
Klingender, R. C., 2008, “Handbook of Specialty Elastomers,” CRC Press
Ionescu, M, 2016, “Chemistry and Technology of Polyols for Polyurethanes, 2^{nd} Edition, Volume 1,” Smithers
Prisacariu, C., 2011, “Polyurethane Elastomers: From Morphology to Mechanical Aspects,” Springer
Thomas, S., Mathew, Chandra, A. K., and Visakh, P. M., 2012, “Advances in Elastomers I: Blends and Interpenetrating Networks (advanced Structured Materials),” Springer

### TECHNICAL FIELD OF THE INVENTION

The present invention relates to polyurethane-urea elastomers with exceptional mechanical properties. More particularly, the present invention pertains to a polyurethane-urea elastomer with ultra-high elongation-at-break and good recovery prepared using a combination of: (1) polyols with ultralow unsaturation and narrow polydispersity, (2) diisocyanates with symmetric steric conformation, (3) bulky diamines between functional groups without steric disruption, and (4) avoidance of chain termination or branching. The preparation method for the novel polyurethane-urea is also disclosed in the present invention. The subject elastomers have the highest reported elongation-at-break among polyurethane-urea elastomers.

### BACKGROUND OF THE INVENTION

Current development of high mechanical performance polyurethane elastomers rely on the usage of polyols synthesized using double metal cyanide complex (DMC) catalysts. Recent advances in the development of DMC catalysts [U.S. Pat. No. 5,712,216, U.S. Pat. No. 5,952,261, U.S. Pat. No. 6,204,357, U.S. Pat. No. 6,291,388] have enabled the synthesis of novel PPG polyols with ultralow unsaturation and polyether polyols, polyether-co-polycarbonate polyols and polyether carbonate polyols with narrow polydispersity [U.S. Pat. No. 6,713,599 B1, U.S. Pat. No. 9,080,010 B2]. These low cost, commercial polyether polyol, polyether-co-polycarbonate polyol and polyether carbonate polyols are commonly used to produce newer polyurethane elastomers with improved physical properties. Polyurethane and polyurethane-urea elastomers with excellent mechanical properties have been developed using polytetramethylene ether glycol (PTMEG) or polyether polyol, prepared using DMC catalysts, to improve physical properties [U.S. Pat. No. 5,648,447, U.S. Pat. No. 5,691,441, U.S. Pat. No. 5,696,221, U.S. Pat. No. 5,708,118, U.S. Pat. No. 5,723,563, U.S. Pat. No. 5,843,357, U.S. Pat. No. 5,948,875, U.S. Pat. No. 6,420,445, U.S. Pat. No. 6,586,566, U.S. Pat. No. 6,624,281, U.S. Pat. No. 6,737,497, U.S. Pat. No. 6,824,703, U.S. Pat. No. 6,903,179, U.S. Pat. No. 6,906,163, U.S. Pat. No. 7,045,650, U.S. Pat. No. 511,111, U.S. Pat. No. 8,273,848]. However, an important limitation of polyurethane elastomers is that it has heretofore been impossible to obtain a high tensile strength polyurethane-urea elastomer with elongation-at-break of over 1350% [U.S. Pat. No. 5,723,563].

Elastomers with the highest reported elongation at break (%) are as follows: natural rubber (NR, cis-1,4-polyisoprene): 1,300%; polybutadiene (BR): 850%; styrene/butadiene/styrene (SBS): 1,750%; thermoplastic polyurethane: 1,400%; polyurethane-urea: 1,350% [U.S. Pat. No. 5,723,563]; conventional silicone elastomer: 1,200% and ultra-high elongation silicone nanocomposite elastomer: 5,000% [Goff]. While silicone elastomers can achieve higher levels of elongation, they also require complicated manufacturing processes and expensive raw materials.

For the synthesis of elastomer prepolymers, aromatic diisocyanates such as 4,4′-methylene diphenylene diisocyanate (MDI), 1,5-NDI, TDI or aliphatic diisocyanate IPDI are commonly used. However, these asymmetric diisocyanates will not form compact urethane links, adversely affecting physical properties. Asymmetric diisocyanate MDI has a twisted steric conformation with a single CH₂ group between two aromatic rings. These rings are difficult to rotate due to steric hindrance, and thus the MDI steric conformation is rigid and asymmetric. TDI contains 65% 2,4-TDI which is asymmetric. IPDI has a bulky asymmetric steric conformation and its isocyanate isomers form cis-cis, cis-trans, and trans-trans orientations, leading to rigid steric conformations. Use of MDI, 2,4-TDI and IPDI diisocyanates increase the difficulty to form urethane links with compact hard segments; thus, the long-range connectivity of the hard segments will also decrease due to the disruption of crystalline regularity, which in turn reduces a percolation of the hard phase through the soft matrix [Prisacariu] and reduces formulation flexibility.

Many recent inventions use a diol chain extender to form urethane links. Compared to urea, urethane provides weaker hydrogen bonding strength. It is known that diamine chain extension increases hydrogen bonding strength with polyurethane-urea links, improving physical properties. Recent inventions disclosing polyurethane-ureas with improved elongation properties have used the following diamine chain extenders: EDA, 1,3-PDA, IPDA, MCDEA. However, EDA is a very small diamine and cannot expand hard segment length. 1,3-PDA and 1,3-diaminopentane have very short distances between amine functional groups with an odd carbon chain between amine groups. This configuration causes significant strain and instability for physical crosslinking, leading to poor physical properties. IPDA has a bulky asymmetric steric conformation and its diamine isomers form cis-cis, cis-trans, and trans-trans orientations, leading to rigid steric conformations. MCDEA has a twisted steric conformation with a single CH₂ group between two aromatic rings. Due to steric hindrance, both IPDA and MCDEA increase the difficulty to extend compact hard segments and decrease long-range connectivity of the hard segments.

Bulky diamines, such as 2-methyl-1,5-pentanediame (2MPDA), 2,2-dimethyl-1,3-diaminopentane or their blends, are used as chain extenders for polyurethane-urea spandex fibers [U.S. Pat. No. 4,973,647, U.S. Pat. No. 5,000,899, U.S. Pat. No. 5,981,686, U.S. Pat. No. 6,720,403] to improve heat setting by extending hard segments. The prepolymers for these polyurethane-urea spandex fibers were synthesized using: (1) a mixture of PTMEG MW1800 and PTMEG MW2000 reacted with MDI [U.S. Pat. No. 4,973,647], (2) PTMEG (PolyTHF/PolyMTHF) reacted with MDI [U.S. Pat. No. 5,000,899], (3) PTMEG MW 1800 reacted with MDI [U.S. Pat. No. 5,981,686], and (4) polymeric glycols reacted with MDI and ortho-substituted MDI [U.S. Pat. No. 6,720,403]. However, MDI cannot form compact hard segments and the PTMEG used in these inventions have high unsaturation with high monol contents. Thus, the resulting polyurethane-ureas have low physical properties including low ultimate elongation.

Other inventions, such as U.S. Pat. No. 6,906,163, blend diethyl amine (DEA) into diamine chain extenders. Chain terminators, such as DEA, reduce polyurethane-urea molecular weight, leading to shorter polymer molecules. Their usage is undesirable since achieving ultra-high elongation requires the molecular weight of the polymer to be as high as possible. Additionally, many other inventions conduct polyurethane-urea synthesis under conditions favorable for polymer chain branching, which also leads to inferior elastomeric properties.

There exists a need for an inexpensive polyurethane-urea with ultra-high elongation and good recovery. These elastomers are useful for rain and sand erosion resistance, wear resistance, tear and puncture resistance, impact attenuation and other forms of surface protection, as well as material reinforcement, among many other usages. Possible application fields are very broad, encompassing transportation, rubber, automotive, textile, packaging, appliances, and healthcare.

### SUMMARY OF THE INVENTION

The present invention is directed to polyurethane elastomers prepared by diamine chain extension of an isocyanato-terminated prepolymer. The present invention overcomes drawbacks inherent in the prior art by providing novel compositions and methods for economically producing polyurethane elastomers with exceptional physical properties including ultra-high elongation-at-break, excellent abrasion resistance, UV and weathering resistance, hydrolysis resistance, and good hardness, modulus, and compression.

The preferred methods of the invention generally comprise the synthesis of an isocyanato-terminated prepolymer in the presence of a catalyst, followed by chain extension of the prepolymer with a diamine.

In one embodiment, an isocyanato-terminated prepolymer composition for producing a polyurethane elastomer exhibiting ultra-high elongation-at-break is disclosed. The prepolymer comprises (1) a polyol having a number average molecular weight around 1,000-8,000 Da. with a significant amount (greater than 80% by weight, based on total weight of polyol component) of diol with ultralow monol content and narrow polydispersity; and (2) a diisocyanate with symmetric molecular structures.

In one embodiment, a diamine composition for the chain extension of the isocyanato-terminated prepolymer is disclosed. The diamine composition comprises a significant amount (greater than 99.0% by weight, based on total weight of diamine component) of a diamine having bulky molecular structures between functional groups.

The elastomers produced in accordance with the present invention are most preferably synthesized using a NCO-capped prepolymer formed by the reaction between a polyol and excess diisocyanate. The molar ratio of the NCO functional group of the diisocyanate to OH functional group of the polyol should be greater than 2.0. For producing elastomers with excellent physical properties, chain extension by a diamine is preferred. The NCO-terminated prepolymer is chain extended by a diamine to form the final polyurethane elastomer.

### DETAILED DESCRIPTION OF THE INVENTION

An elastomer is a type of polymer with viscoelasticity, weak intermolecular forces, high ultimate elongation and low Young's modulus. Ultimate elongation or elongation at break is a measurement of elongation at the point in which a sample breaks under tension. It is well known that the elasticity of natural rubber originates from a preponderance of wrinkled conformations over more linear conformations. For synthetic triblock copolymers, such as thermoplastic elastomers, “physical crosslinking” of hard domains entangled within a soft amorphous hydrocarbon matrix provide elasticity [Drobny, Hadjichristidis]. Triblock ABA copolymers are composed of phase segregated hard domains (polystyrene), which are regularly arranged in a soft matrix (polyisoprene or polybutadiene) [Drobny, Hadjichristidis]. Crystallinity, hydrogen bonding, and van der Waals interaction of hard segments all lead to microphase separation, thus affecting elasticity. For both natural and synthetic elastomers, having a very long molecule with high molecular weight is critical for providing excellent elasticity [Balkazara, Klingender].

According to convention, “polyurethane” encompasses both polyurethanes and polyurethane-urea. Polyurethane is a polymer characterized by carbamate (urethane) (—NH—CO—O—) links, which are formed by reactions between isocyanato (—N═C═O) groups and hydroxyl (—OH) groups. Polyurea is a polymer characterized by urea links (—NH—CO—NR—), which are formed by reactions between isocyanato (—N═C═O) groups and amino (—NHR—) groups. Urethane and urea links are desirable because both bonds are chemically stable and provide hard crystalline domains. These hard segments (HS) (crystalline domain) are immiscible and separate from soft segments (SS) (soft matrix of polyether, polyester, or polycarbonate). The elastic properties of the resulting polymer are directly related to the degree of microphase separation [Prisacariu, Thomas].

Polyurethane-ureas produced by step-growth polymerization are used extensively for a variety of commercial applications [Clemitson, Prisacariu]. A silicone elastomer nanocomposite has been recently developed with ultra-high ultimate elongation using this method. It is composed of a very high molecular weight silicone, formed by step-growth polymerization, combined with fumed silica. Its elasticity originates from constrained inter- or intra-chain entanglements of linear polydimethylsiloxane within the nanocomposite [Goff].

Microphase separation generally occurs in segmented polyurethanes, polyurea, and polyurethane-urea due to thermodynamic incompatibility resulting in the insolubility of hard segments within the soft segments. Soft segments typically are composed of a relatively long, flexible polyester or polyether diol with a molecular weight between 1,000-4,000 Da. (Daltons). They are termed soft segments because they impart softness and flexibility to materials as they are non-crystalline at usage temperatures. Additionally, their T_{g} (glass transition temperature) are below the lower usage temperature of the polymer, resulting in a rubbery material. The hard segments are typically extended by the reaction of diisocyanates with diol or diamine chain extender (CE) molecules, for example 1,4-butanediol and 1,4-diaminobutane. The resulting hard segments serve to increase hydrogen bonding, resulting in higher elongation, tensile strength, and hardness, but lower recovery. A key characteristic of hard segments is their polar nature due to the urethane and urea groups they contain. These groups can form intra-, or particularly, intermolecular hydrogen bonds with ether or ester soft segments and with other urethane/urea groups. When bonding with other urethane/urea groups, they can segregate themselves into domains rich with hard segments. Hence, a microphase separated morphology develops with interdomain spaces varying from a scale of ca. 30 Å-100 Å. In addition to microphase separation due to the incompatibility of hard and soft segments, crystallization of the hard segments can also be a driving force for microphase separation.

Low levels of hard segment (HS) content often produce either 1) mixed systems (since in typical systems, microphase separation is a function of HS-SS incompatibility due in part to HS segment length) and/or 2) microphase separated structures which contain HS domains that are dispersed throughout a SS matrix with little interaction. The resulting weak domain structure leads to soft materials with poor recoverability. As the HS content is increased by lengthening the HS (generally by the addition of a chain extender and additional diisocyanate), a more desirable microphase separated morphology can develop. With higher HS content, the long range connectivity of the HS also increases until it approaches the range of around 30 wt %, which results in an interconnected hard phase. The percolated structure increases both tensile strength and hardness, although such interconnected structures suffer from limited recoverability following elongation.

Linear polyurethanes are synthesized using a step-growth reaction technique first developed by Otto Bayer in the late 1930s. With the more commonly used prepolymer method, linear hydroxyl terminated oligomeric polyether or polyesters are reacted with excess diisocyanate to cap the oligomer, thereby forming a urethane linkage and leaving an isocyanate functional group at each terminus, resulting in what is termed a “prepolymer”. This prepolymer mixture (containing excess diisocyanate) is then reacted with a diamine chain extender to form hard segments and increase the molecular weight of the macromolecule. In general, an increase in HS content leads to increased modulus (stiffness) and enhanced tensile strength.

Segmented polyurethanes are generally synthesized using the prepolymer method. A less common synthesis method for elastic polyurethane is a one-step process in which the polyol, diisocyanate, and chain extender are simultaneously mixed together to form the final product. In both cases, a chain extender is used to increase HS content and to modify the physical properties. [Clemitson, Prisacariu].

The present invention is based on studies of segmented polyurethane-urea synthesized using the prepolymer and chain extender method to promote a desired microphase separated morphology for achieving extremely high elasticity. Before the present invention is described in further detail, it is to be understood that the invention is not limited to the particular embodiments described, as such may, of course, vary, it is also to be understood that the terminology used herein is for the purpose of describing particular embodiments only, and is not intended to be limiting, since the scope of the present invention will be limited only by the appended claims.

By increasing the molecular weight of the polyol, the unsaturation or monol content can be greatly increased in conventional polypropylene glycols (PPGs) synthesized using potassium hydroxide catalysts. For example, a 2,000 Da. conventional PPG typically has an unsaturation content of 0.030 meq/g which corresponds to a functionality of 1.94 (monol: 12.5 mole %), whereas a 4,000 Da. diol has an unsaturation content of 0.085, and functionality of 1.69. This mixture is approximately 70% diol and 30% monol on a mole percentage basis. Usage of a DMC (double metal cyanide complex) catalyst to produce polyether polyol dramatically lowers the unsaturation content and increases functionality. For example, Acclaim® 4200 (MW 4,000 Da.) has a monol content of 0.005 meq/g or less and a functionality of 1.98 (monol: 3 mole %) versus a conventional PPG, which has an unsaturation content of 0.085 meq/g and functionality of 1.69 (monol: 30 mole %). A measure of the polydispersity of a polymer is the weight average of the molecular weight divided by the number average of the molecular weight. Currently, DMC catalysts can produce PPG commercial products with a polydispersity of less than 1.10.

Polyurethane, polyurea, and polyurethane-urea elastomers have relatively low molecular weights compared to natural rubber or triblock copolymer thermoplastic elastomers. The polyether polyols contribute to the soft matrix of polyurethane-urea elastomers. The diols of polyether polyol provide the linkage to a polyurethane linear chain through polyether polyol terminal hydroxyl groups at both end positions. With the hydroxyl group at one end, the monol terminates the polyurethane chain. By increasing the molecular weight of polyurethane-urea, we can improve its mechanical properties. The single most important factor contributing to the performance of polyurethane is its monol content; since monols terminate polyurethane chains, they can dramatically reduce the molecular weight of polyurethane. In order to maximize molecular weight in the present invention, polyols with ultralow unsaturation content are preferred. Additionally, polyols with narrow polydispersity are also preferred for the synthesis of prepolymers.

The first step of this invention involves prepolymer synthesis, wherein a symmetric diisocyanate is selected to form an isocyanato terminated prepolymer. Diisocyanate symmetry was found to play a key role in the performance characteristics of the resulting polymer, with symmetric diisocyanate leading to better microphase separation with compact hard segments. Isocyanato-terminated prepolymers are formed by the reaction between the polyether polyol component (with ultralow unsaturation and narrow polydispersity) with the excess symmetric diisocyanate component. The molar ratio of the NCO functional group of diisocyanate to the OH functional group of the polyol is greater than 2.0, leading to a prepolymer with ultra-high functionality. The resulting prepolymer has compact hard segments due to the symmetric molecular structure of diisocyanate and displays good microphase separation. When UV and weathering resistant is required, an aliphatic or cycloaliphatic symmetric diisocyanate can be selected.

Hydrogen bonding is an attractive force between a lone pair of electrons of an electronegative atom such as N or O and a hydrogen atom that is directly attached to an N or O. Hydrogen bonding between an electronegative O and a single H that is attached to an N is termed monodentate hydrogen bonding. Hydrogen bonding between an electronegative O and two H atoms attached to two different N atoms is termed bidentate hydrogen bonding. The strength of hydrogen bonding in a polyurethane chain has a direct effect on the final properties of these materials. Hydrogen bonding in a urethane link is monodentate while hydrogen bonding in a urea link is bidentate.

During the second step of the present invention, a polyurethane-urea is formed by reacting the isocyanato-terminated prepolymer with stoichiometric amounts of bulky bifunctional diamine chain extender with urea links. Since bidentate hydrogen bonding is stronger than monodentate hydrogen bonding, a chain extended by a diamine with urea links will have longer polyurethane-urea hard segments than a chain extended by a diol with urethane links. Thus, selecting a bulky diamine to serve as a chain extender can enlarge hard segment sizes. As an additional benefit, polyurethane-urea materials display a broader range of service temperature windows than polyurethane chain materials extended with diol counterparts due to their stronger bidentate hydrogen bonds.

Asymmetry of the chain extender disrupts hard segment regularity and interferes with hard segment intermolecular association; consequently, poor physical properties are obtained. Bulky diamines, though asymmetric, display intermediate behavior due to high chain stiffness and reduced chain flexibility. In general, aromatic diamines that possess bulky and symmetric structures will give optimum properties. However, when UV and weathering resistant is required, an aliphatic or cycloaliphatic bulky diamine is required.

The relationship between chemical structure and mechanical properties of linear segmented polyurethane-urea is explored in the present invention to obtain an elastomer with ultra-high ultimate elongation. Soft segments generally have lower molecular weights (less than 8,000 Da.) than anionic ABA block copolymers (often greater than 150,000 Da.). In polyurethane, the soft segments (SS), such as polyether units, have glass transition temperatures (T_{g}) well below room temperature. The hard segments (HS) of polyurethane-urea are composed of urethane units and chain extended by diamine moieties to increase HS length and thereby the mass of the HS. Typically, chemical incompatibility between soft and hard segments increases as segment lengths increase. This incompatibility drives the microphase separation process, leading to a service temperature window often defined by the SS T_{g} and upper temperature softening transition of the HS domains. The service temperature window typically lies above the SS T_{g} and below the softening point of the hard segments or the polyurethane-urea degradation temperature. More specifically, the upper temperature limit is bound by one of three following conditions: 1) the hard segments softening at the HS T_{g}, 2) the hard segments melting due to a crystalline HS at Tₘ, or 3) the material degrading at high temperatures prior to softening. For a higher service temperature window, it is desirable to form microphase separation of hard segments of urethane links extended by urea links dispersed in a continuous amorphous SS matrix.

If the relative hard segment volume or weight fraction of the polyurethane is low, the HS is generally postulated to segregate into isolated microdomains that are randomly dispersed throughout a continuous matrix of the soft segment phase. As the HS content is increased, the long-range connectivity of the HSs is also expected to increase, which in turn promotes a percolation of the hard phase through the soft matrix. The hard domains act as physical crosslinking sites and reinforce the soft matrix, thereby enabling the copolymer to display controllable structural properties within its service window, the region often referred to as a “plateau region”. In addition to the HS content, the extent of the HS percolation and the potential crystalizing ability of the hard phase can also greatly influence the mechanical and thermal response of the material. In segmented polyurethane, polyurea and polyurethane-urea, the cohesiveness of the hard domains is further enhanced by the ability of the HS to establish a hydrogen bonded network, particularly within the HS phase.

In one embodiment of present invention, the polyurethane-urea is preferably prepared by chain extension of an isocyanato terminated prepolymer with a diamine chain extender. The isocyanato terminated prepolymer is prepared by reacting diisocyanate with a polyol component. The polyol component preferably has number average molecular weight ranging from 1,000 to 8,000 Da. The polyol component is preferably selected from polyols having ultralow unsaturation content (very low monol content) and narrow polydispersity. Polyether polyols can be economically synthesized from propylene oxide using DMC catalysis. Recently developed DMC applications can provide polyether-co-polycarbonate polyols (polyether polycarbonate polyols) with ultralow unsaturation and narrow polydispersity.

It is known that polyurethanes synthesized using polyether polyols are more chemically stable and hydrolysis resistant than polyurethanes synthesized using polyester polyols. The cohesive energy between ester groups of polyurethanes synthesized using polyester polyols or polycarbonate polyols are greater than those synthesized using polyether polyols. Increases in cohesive energy corresponds to higher tensile strength. However, commercially available polyester polyols do not possess the ultralow unsaturation content found in polyether polyols synthesized using DMC catalysts. Therefore, polyether polyols or polyether-co-polycarbonate polyols produced using DMC synthesis are preferred.

Since low unsaturation provides high functionality, long chain polyurethane-urea molecules and improved physical properties, to obtain polyurethane-ureas with ultra-high elongation, polyether polyols with ultralow unsaturation and narrow polydispersity are preferred for the synthesis of the isocyanato terminated prepolymer. Selection of the molecular weight for a preferred polyether polyol is affected by the following considerations: (1) to produce a polyurethane-urea with strong, durable mechanical properties, the final molecular weight must be greater than 5×10⁴ Dalton (for comparison, natural rubber latex has a molecular weight of 5×10⁶ and an ABA triblock copolymer elastomer has an approximate molecular weight of 1.5×10⁵ Dalton); (2) the elasticity of polyurethane-urea elastomers synthesized using low molecular weight polyether polyols as expressed by ultimate elongation is relatively low; (3) prepolymers and polyurethane-urea synthesized using high molecular weight polyether polyols or chain branching using triols have a higher viscosity than those produced using low molecular weight linear polyether diols, and (4) high viscosity has undesirable effects, including poor control during processing, tendency to trap air bubbles, and difficulties in mixing and transport. Highly viscous formulations require lowering of the solid percentage by dilution with solvent to make the isocyanato terminated prepolymer or final polyurethane-urea solution processable. High solvent percentages increase production costs associated with solvent recovery and vacuum equipment capacity. Thus, there is a tradeoff between molecular weight and cost of production for polyether polyols or polyether-co-polycarbonate polyols for synthesizing polyurethane elastomers.

In the present invention, for synthesizing polyurethane-urea with ultra-high elongation-at-break, higher molecular weight polyether diols or polyether-co-polycarbonate diols with at least a number average molecular weight of 2,000 to 4,000 Daltons is required and a modern polyurethane casting process is preferred.

The rheological behavior of a polymer system is profoundly influenced by its molecular weight, molecular weight distribution, and polydispersity (weight average molecular weight divided by number average molecular weight). Viscosity and shear rate are sensitive to polymer polydispersity, especially within the high molecular weight region. A mixture of polyols with broad polydispersity (broad molecular weight distribution) will show high viscosity and shear rate. It is well known that resins required for high solid applications need to have a narrow molecular weight distribution (low polydispersity) since solution viscosity is strongly influenced by polymer molecular weight and molecular weight distributions.

Polyurethane is a step growth polymer. The molecular weight distributions of final products are much broader than the distributions of the initial polyols used to prepare the prepolymers. Narrower initial polyether polyol molecular distributions and careful selection of synthesis conditions will provide for relatively narrower polydispersity of the final polyurethane-urea molecular weight distribution. To produce a polyurethane-urea elastomer with ultra-high elongation at break, a very careful selection of polyether polyol or polyether carbonate polyol is required. The ideal polyol is a polyether diol or polyether-co-carbonate diol with ultralow unsaturation and narrow polydispersity. Therefore, a polyether diol or polyether-co-polycarbonate diol (or diol blend) should be selected with a single value for average molecular weight (either number average or weight average), not a blend of diols with different number averages or weight averages for molecular weights.

For example, polytetrahydrofuran, (polytetramethylene ether) glycol (PolyTHF® by BASF, PTMEG) is a polyether diol, but with relatively higher unsaturation. It is a waxy solid at ambient temperature. The prepolymer is synthesized using a PTMEG with a molecular weight around 2,000 Dalton and MDI (diphenylmethane diisocyanate) with an extremely high viscosity. The final product, spandex, does not have a high elasticity due to the lower unsaturation and polydispersity of PTMEG [U.S. Pat. No. 4,973,647, U.S. Pat. No. 5,000,899, U.S. Pat. No. 5,981,686, U.S. Pat. No. 6,720,403].

In one embodiment of the present invention, preferred polyether diols with ultralow unsaturation and narrow polydispersity are used for the synthesis of an isocyanato terminated prepolymer. Commercially available polyol diols obtained using DMC catalyst processes have molecular weights between 1,000 to 8,000 Daltons, including Acclaim® 2200 (MW 2,000), Acclaim® 2220N (MW 2,250), Acclaim® 4,200 (MW 4,000), and Acclaim® 4220 (MW 4,000), Acclaim® 8200 (MW 8,000) from Covestro.

Triols are useful to promote branching of polyurethane-urea, which increases tensile strength and wear and abrasion resistance, but reduces elasticity. Examples of commercial triols with ultralow unsaturation are: Acclaim® 3300N (MW 3,000), Acclaim® 6300 (MW 6,000), and Acclaim® 6320N (MW 6,000) from Covestro. When higher tensile strength and hardness is required, a polyurethane-urea elastomer with less elastic properties can be produced by mixing a smaller proportion of triols to provide chain branches.

Use in outdoor field applications, such as aviation, wind turbines, vehicles, marine, and construction requires long service life with UV and weathering resistance. Therefore, for these applications, it is highly desirable to utilize aliphatic compositions due to their proven performance in hostile UV and weathering environments.

In one embodiment of the present invention, a steric symmetric diisocyanate is preferred for the synthesis of the isocyanato terminated prepolymer. Symmetric diisocyanate forms isocyanato terminated prepolymers with compact polyurethane hard segments and leads to better microphase separation, long-range connectivity of the HS, and percolation of the hard phase through the soft matrix to build a hydrogen bonding network. Isocyanato terminated prepolymer can be synthesized by the reaction between a polyether polyol component with ultralow unsaturation and narrow polydispersity with excess steric symmetric diisocyanate. Examples of steric symmetric diisocyanate that can be used for the present invention include, but are not limited to: (1) aliphatic diisocyanate: hexamethylene diisocyanate (HDI); (2) cycloaliphatic diisocyanate: trans-para-cyclohexyl diisocyanate (t-1,4-cyclohexylene diisocyanate) (t-CHDI), trans,trans-4,4′-dicyclohexyl-methylene diisocyanate (4,4′-diisocyanato dicyclohexylmathane) (t,t-H₁₂MDI); and (3) aromatic diisocyanate: p-phenyl diisocyanate (1,4-phenylene diisocyanate) (PPDI), 4,4′-dibenzyl diisocyanate (DBDI), 2,6-toluene diisocyanate (2,6-TDI), and 1,5-naphthalene diisocyanate (1,5-NDI). Preferred commercially available diisocyanates include HDI, PPDI, DBDI, 2,6-TDI, and 1,5-NDI.

Other diisocyanates are commercially available, but provide polyurethane-urea with relatively low elasticity. Examples include 2,2,4-trimethyl-1,6-hexamethylene diisocyanate (TBDI), asymmetric aliphatic diisocyanate, trimethyl-1,6-diisocyantohexane (mixture of 2,2,4-trimethyl-hexamethylene-diisocyante and 2,4,4-trimethyl-hexamethylene diisocyanate), 4,4′-dicyclohexyl-methylene diisocyanate (hydrogenated 4,4′-diphenylmethane diisocyanate) (H₁₂MDI) which has three geometrical isomers: trans-trans, cis-trans, and cis-cis, asymmetric cycloaliphatic diisocyanate, 1-isocyanto-3-isocyanatomethyl-3,5,5-trimethyl-cyclohexane (isophorone diisocyanate) (IPDI), asymmetric cycloaliphatic diisocyanate mixture: methyl-cyclohexamethylene-diisocyanate) (HTDI) having 2,4-HTDI and 2,6-HTDI isomers, toluene diisocyanate (TDI) having two isomers (2,4-TDI and 2,6-TDI), 4,4′-methylene diphenyl diisocyanate, bis(4-isocyanatophenyl)methane (MDI), and xylylene diisocyanate (XDI) having two isomers (2,4-XDI and 2,6-XDI).

In one embodiment of the present invention, isocyanato terminated prepolymers can be synthesized by a reaction using: (1) a polyol component having a number average molecular weight ranging from 2,000 to 8,000 Daltons and (2) a diisocyanate component. The polyol component must include: (1) a significant amount (greater than 80% by weight, based on total weight of polyol component) of diol with ultralow monol and narrow polydispersity having a number average molecular weight ranging from 2,000 to 4,000 Daltons. The diisocyanate component must include a significant amount (greater than 99.5% by weight, based on total weight of diisocyanate component) of diisocyanate having symmetric molecular structures and high functionality.

In one embodiment of the present invention, the preferred polyol component of the prepolymer is a single variety of polyether diol with a number average molecular weight concentrated at a single value between 2,000 to 4,000 Daltons, with ultralow unsaturation and narrow polydispersity. In the same embodiment, the preferred diisocyanate component of the prepolymer is a single variety of symmetric diisocyanate with high functionality and high purity. Additionally, in the same embodiment, the molar ratio of the NCO functional group of diisocyanate to OH functional group of the polyol is greater than 2.0 (NCO/OH of diisocyanate over diol).

In another embodiment of the present invention, the preferred polyol component of the prepolymer is a single variety of polyether-co-polycarbonate diol or polyether carbonate diol with a number average molecular weight concentrated at a single value between 2,000 to 4,000 Daltons, with ultralow unsaturation and narrow polydispersity. In the same embodiment, the preferred diisocyanate component of the prepolymer is a single variety of symmetric diisocyanate with high functionality and high purity. Additionally, in the same embodiment, the molar ratio of the NCO functional group of diisocyanate to OH functional group of the polyol is greater than 2.0 (NCO/OH of diisocyanate over diol).

In one embodiment of the present invention, the prepolymers may be prepared using any conventional techniques. The present invention also provides a method for prepolymer synthesis. Since polyether polyols are slightly hydroscopic and absorb water, the present invention provides a method for the dehydration of the polyether polyol component: apply a nitrogen blanket and heat the polyether polyol component in a reactor under vacuum to remove all traces of water to avoid interference from water. The terminal hydroxyl groups of polyether polyols are secondary hydroxyl groups and have lower reactivity than primary hydroxyl groups. To produce a linear prepolymer without chain branching under lower reaction temperatures, a catalyst is required. A homogeneous catalyst, such as a tertiary amine or organometallic complex, at a concentration of 0.001 w/w % (10 ppm) to 0.05 w/w % (500 ppm) can be added to accelerate the reaction between hydroxyl groups and isocyanato groups in the reaction system. After cooling the polyether polyol component to ambient temperature, a diisocyanate component is added into the reactor with the dehydrated polyether polyol component under a nitrogen blanket and vigorous stirring. The molar ratio of the NCO functional group of diisocyanate to the OH functional group of polyether polyol is preferably between 2.05 to 2.10. The inventors of the present invention conducted prepolymer synthesis by heating a mixture of the polyol component and diisocyanate component under a nitrogen atmosphere at a temperature range between 40° C. to 80° C. and achieved complete reactions with all terminal hydroxyl groups of the polyols replaced by diisocyanate terminated prepolymer.

Metal containing compounds such as polyaddition catalysts are known in the art. It is well known that polyaddition involving hydroxyl and isocyanato can be accelerated by a variety of metals, metal oxides, metal complexes, and organometallics. Noble metals and their complexes are the most active but also most expensive, such as platinum, rhodium and ruthenium complexes. Organomercury compounds, such as phenylmercuric acetate are exceptionally active and selective catalysts for hydroxyl and isocyanato to form urethane groups. Organolead compounds also have high catalytic activity. However, both organomercury and organolead are not preferred due to their high neurotoxicity and dangerous environmental profile. Thus, other metal complexes are preferred as catalysts for controlling the polyaddition reaction between isocyanato and hydroxyl or between isocyanato and a secondary amine group. Examples of metal elements include, but are not limited to, bismuth, calcium, cerium, cobalt, copper, iron, lithium, manganese, silver, tin, titanium, zinc, and zirconium. Various mercaptans can provide long induction times when used in conjunction with bismuth/zinc carboxylate.

Preferred catalysts in the present invention include, but not limited to, aluminum dionate, aluminum octoate, aluminum tris(2,4-pentanedionate), bismuth carboxylate; amine-cuprous chloride, calcium octoate, iron octoate; silver oxide, silver nitrate, silver, nitrite, tin carboxylate, tin octoate, tin II neodecanoate, tin II octoate, tin II oleate, dibutyltin diacetate, dibutyltin dilaurate, dibutyltin diisooctylmaleate, dimethyltin dineodecanoate, dioctyltin dilaurate, bis(2-ethylhexanoate)tin, stannous octoate, bis(2-ethylhexanoate)tin, bis(neodecanoate)tin, di-n-butyl bis(2-ethylhexylmaleate)tin, di-n-butyl bis(2,4-petanedionate)tin, di-n-butylbutoxychlorotin, di-n-butyldiacetoxytin, di-n-butyldilauryltin, di-n-butyldilauryltin, dimethyldineodecanoatetin, dimethylhydroxy(oleate)tin, dioctyldilauryltin, titanium di-n-butoxide, titanium (bis-2,4-pentanedionate), titanium diisopropoxide, titanium 2-ethylhexoxide, titanium (bis-2,4-pentanedionate), titanium diisopropoxide, titanium bis(ethylacetoacetate), titanium 2-ethylhexoxide, titanium trimethylsiloxide, zinc octoate, zirconium dionate, zirconium 2-ethylhexanenoriate, and zirconium tetrakis(2,4-pentanedionate) complex. The amount of an organometallic catalyst needed is based on the total weight of the polyether diol, preferably ranging from 0.001 w/w % (i.e. 10 ppm) to 0.02 w/w % of the total weight of the polyether diol.

Organic catalysts, such as tertiary amines, cyclic guanidine and amidines are also very active catalysts for polyaddition of hydroxyl groups with isocyanato groups. Most organic catalysts are composed of 1,4-diazabicyclo[2,2,2]octane (DABCO), 1,8-diazabicyclo[5,4,0] undec-7-ene (DBU), 1,5,7-triazobicyclo[4,4,0]dec-5-ene (TBD), N-methyl-1,5,7-triazobicyclododecene (MTBD), diethanolamine, 3-dimethylamino-N,N-dimethylpropioamide, bis[2-dimethylaminoethyl]ether, N-[2-(dimethylamino)ethyl]-N-methylethanolamine, 2[2-dimethylaminoethyoxyl]ethanol, 3-dimethylamino-N,N-dimethylpropioamide, N,N-dimethylcyclohexylamine, dimethylethanolamine, N-ethylmorpholine, N,N,N′,N′,N′-pentamethyldiethylenetriamine, tertiary phosphines, tetrachlorocuprate having anilinium, p-methoxyanililinum, p-hethylanilium and pyridium cations, triethylamine, triethylenediamine, and a mixture thereof. However, organic catalysts are less selective than organometallics and require a greater quantity of catalyst to achieve a similar effect.

In the present invention, the isocyanato terminated prepolymer is chain extended by a diamine component to (1) provide a urea link unit, (2) increase hard segment length, and (3) link isocyanato terminated prepolymers together to form a high molecular weight macromolecular polyurethane-urea. The diamine component must include a significant amount (greater than 99.0% by weight, based on total weight of diamine component) of diamine having bulky molecular structures.

In one embodiment of the present invention, the preferred diamine chain extender is a bulky diamine having at least a four-carbon chain between attached amino groups; examples include, but are not limited to: (1) bulky symmetric aliphatic diamines or bulky asymmetric aliphatic diamines, such as: 1,4-diaminobutane, 1,5-dimanopentane, 1,6-diaminohexane, 2-methyl-1,5-diaminopentane, 3-methyl-1,5-diaminopentane, 2-methyl-1,6-diaminohexane, 3-methyl-1,6-diaminohexane, 1,8-diaminooctane, 1,10-diaminodecane, and 1,12-diaminododecane; (2) bulky symmetric steric specific cycloaliphatic diamines, such as: trans-1,4-cyclohexyl diamine (t-CHDA), trans-1,8-diamino-p-menthane, and trans,trans-4,4′-dicyclohexyl-methylene diamine (t,t-H₁₂MDA); (3) bulky asymmetric diamines, such as: 2,2-dimethyl-1,3-propanediamine (neopentadiamine); (4) bulky symmetric aromatic diamines, such as: 4,4-diamino-dibenzyl (DAB), p-phenyl diamine (1,4-phenylenediamine) (PPDA), 1,5-naphthalene diamine (1,5-NDA), 2,6-toluene diamine (2,6-TDA), and p-xylylenediamine; (5) bulky asymmetric cycloaliphatic secondary diamines, such as: 1-isopropylamino-3-methylene isopropylamino-3,5,5-trimethyl-cyclohexane (Jefflink 754), 4,4′-dibutylaminodicyclohexyl methane (Clearlink 1000), polyaspartic ester diamine, aldimine, polyalkyl diamine, polyoxypropylene diamine, and (polyethylene glycol) diamine; (6) other bulky aromatic diamines, such as: 2,5-bis(4-amino-phenylene)-1,3,4-oxadiazole (DAPO), and 2,6-diamino-pyridine (DAPY).

Less preferred diamines include (1) bulky asymmetric cycloaliphatic with chirality or twist conformations, which lead to steric hindrance, such as: a mixture of cis and trans isomers of 1,4-cyclohexyl diamine (CHDA), 3-Amino-methyl-3,5,5-trimethyl-cyclohexylamine (isophorone diamine) (IPDA), a mixture of cis and trans isomers of 1,8-diamino-p-menthane, a mixture of cis-trans, cis-cis, and trans-trans-isomers of 4,4′-dicyclohexyl-methylene diamine, and a mixture of 3,3′-dimethyl-4,4′-diaminodicyclohexylmethylene and 4,4′-methylene-bis(2,6-diethylcyclohexaamine) (M-DECA); (2) bulky asymmetric or symmetric aromatic diamine with twist conformation, such as: 4,4′-diaminodiphenylmethane, (MDA), 4,4′-diaminodiphenyl sulfone (DDS), 3,3′-dichloro-4,4′-diaminodiphenyl methane, (MOCA), 1,2-bis(2-aminophenylthio) ethane (Apocure 601E), 3,3′dimethyl-4,4′diaminodiphenyl methane, 1-methyl-3,5-bis(methylthio)-2,6-diaminobenzene, 1-methyl-3,5-diethyl-2,6-diaminobenzene, 4,4′-dimethyl-bis(3-chloro-2,6-diethylaniline), 4,4′-methylene-bis(2,3-dichloroaniline) (4,4′-methylene-bis-(ortho-chloroaniline) (M-DEA), 4,4′-methylene-bis(2,3-dichloroaniline) (M-DCA), 4,4′methylene-bis(2-ethyl-6-methylaniline) (M-MEA), 4,4′-methylene-bis(2,6-diethylaniline), trimethylene bis(4-aminobenzoate) (Polacure 740M); 4,4′methylene-bis(2,6-diisopropylaniline) (M-DIPA), 4,4′-methylene-bis(2-methyl-6-isopropylaniline), 4,4′-methylene-bis(2-isopropylene-6-methylaniline) (M-MIPA), a mixture of 3,5-dimethylthio-2,4-toluenediamine and 3,5-dimethylthio-2,6-toluenediamene (Ethacure 300), a mixture of 3,5-diethyl-2,4-toluenediamine and 3,5-diethyl-2,6-ditoluenediamene (Ethacure 100), 4,4′-methylene-bis(3-chloro-2,6-diethylaniline) (M-CDEA), and methylene-bis(methyl.anthranilate) (MMA).

In the present invention, no chain terminators are allowed. In one embodiment of the present invention, isocyanato terminated prepolymers are reacted with diamine in a solvent or solvent mixture medium under a nitrogen atmosphere. A low concentration of a diamine chain extender in a suitable solvent is added dropwise into an isocyanato terminated prepolymer solution under vigorous stirring. The concentration of diamine in a solvent is preferably kept within the range of 20 w/w % to 1.0 w/w %. The reaction temperature is preferably controlled between −10° C. to 10° C. The molar ratio of the —NH₂ or —NHR functional group of the diamine chain extender to the —NCO functional group of the isocyanato terminal group of the prepolymer must be kept at 1.0. No excess diamine is allowed since it will terminate the polyurethane-urea chains and result in a low molecular weight product with low physical properties.

In another embodiment of the present invention, isocyanato terminated prepolymers are chain extended by a diamine component to form a high molecular weight polyurethane-urea elastomer. The chain extension reactions are carried out in a solvent medium. The solvent must be able to dissolve the reactants of the prepolymer and diamine, as well as the final polyurethane-urea product. Since polar urea links in polyurethane-urea have low solubility, polar solvents provide the best dissolution of polyurethane-urea. Suitable organic solvents for the chain extension medium include, but are not limited to: tetrahydrofuran (THF), dimethyl sulfoxide (DMSO), tetramethylene sulfone (TMS), dimethyl formamide (DMF), dimethyl acetamide (DMAc), N,N-dimethyl ethylene urea, and N-methyl-2-pyrrolidone (NMP). Since primary amino groups are much more reactive than hydroxyl groups, organic alcohols, such as isopropanol, can be used as co-solvents under low temperatures. The solvent or solvent mixture is preferably dried using 3 Å or 5 Å molecular sieves to remove water. The addition of organic alcohols into the polar solvent can reduce cost.

In one embodiment of the present invention, a solvent is selected from the group consisting of acetone, acetonitrile, acetophenone, amyl acetate, benzyl benzoate, butanol, 2-butanol, butanone, butyl acetate, sec-butyl acetate, tert-butyl acetate, gamma-butylolactone, n-butyl propionate, para-chlorobenzotrifluoride, chloroform, cyclobutanone, cyclohexane, cyclohexanone, cyclopentanone, dichloromethane, diethyl carbonate, diethyl ketone, diisobutyl ketone, N,N-dimethylacetamide, dimethyl carbonate, N,N-dimethyl ethylene urea, N,N-dimethylformamide, dimethyl sulfoxide, dioctyl terephthalate, 1,4-dioxane, ethanol, 2-ethoxyethyl ether, ethyl acetate, ethyl acetoacetate, ethyl butyrate, ethyl lactate, ethylene carbonate, bis(2-ethylhexyl) adipate, ethyl isopropyl ketone, hexyl acetate, isoamyl acetate, isobutanol, isobutyl acetate, isobutyl isobutyrate, isopropanol, isopropyl acetate, isophorone, mesityl oxide, methanol, methyl acetate, methyl amyl acetate, methyl butyl ketone, methyl chloroform, methylene chloride, methyl ethyl ketone, methyl isoamyl ketone, methyl isobutyl ketone, methyl isopropyl ketone, methyl propyl ketone, N-methyl-2-pyrrolidinone, octyl acetate, 3-pentanone, n-pentyl propionate, perchloroethylene, propanol, 2-propanol, beta-propyolactone, propyl acetate, tetrahydrofuran, tetramethylene sulfone, toluene, triacetin, delta-valerolactone, xylene, and a mixture thereof.

In the present invention, a polyurethane-urea elastomer exhibiting ultra-high ultimate elongation is disclosed which contains a plurality of urethane/urea hard segments within a polyether soft matrix, wherein said elastomer comprises the reaction product of:
(A) an isocyanato-terminated prepolymer prepared by the reaction of:
   (A1) a diisocyanate with a symmetric molecular structure selected from the group consisting of hexamethylene diisocyanate (HDI); 1,4-cyclohexyl diisocyanate (CHDI), 4,4′-dicyclohexyl-methylene diisocyanate (H₁₂MDI), 1,4-phenylene diisocyanate (PPDI), 4,4′-dibenzyl diisocyanate (DBDI), 2,6-toluene diisocyanate (2,6-TDI), 1,5-naphthalene diisocyanate (1,5-NDI), and a mixture thereof; and
   (A2) a polyether diol having a molecular weight between 2,000 to 8,000 with ultralow unsaturation of monol contents (0.007 meg/g or less) and polydispersity narrower than 1.20; and
   (A3) a homogeneous catalyst for linear polymerization in an amount equal to at least 0.001 w/w % (i.e. 10 ppm);
   wherein the molar ratio of the NCO functional group of said diisocyanate to OH functional group of said polyether polyol is greater than 2.0.
(B) a bulky diamine chain extender having a continuous carbon chain with at least four-carbon atoms between attached amino groups; and
(C) a solvent, to form a solution of said polyurethane-urea.

The present invention also discloses a polyurethane-urea elastomer exhibiting ultra-high ultimate elongation which contains a plurality of urethane/urea hard segments within a polyether-co-polycarbonate soft matrix, wherein said elastomer comprises the reaction product of:
(A) an isocyanato-terminated prepolymer prepared by the reaction of:
   (A1) a diisocyanate with a symmetric molecular structure selected form the group consisting of hexamethylene diisocyanate (HDI); 1,4-cyclohexyl diisocyanate (CHDI), 4,4′-dicyclohexyl-methylene diisocyanate (H₁₂MDI), 1,4-phenylene diisocyanate (PPDI), 4,4′-dibenzyl diisocyanate (DBDI), 2,6-toluene diisocyanate (2,6-TDI), 1,5-naphthalene diisocyanate (1,5-NDI), and a mixture thereof; and
   (A2) a polyether-co-polycarbonate diol having a molecular weight between 2,000 to 8,000 with ultralow unsaturation of monol contents (0.007 meg/g or less) and polydispersity narrower than 1.20; and
   (A3) a homogeneous catalyst for linear step polymerization;
   wherein the molar ratio of the NCO functional group of said diisocyanate to OH functional group of said polyether-co-polycarbonate diol is greater than 2.0.
(B) a bulky diamine chain extender having a continuous carbon chain with at least four-carbon atoms between attached amino groups; and
(C) a solvent, to form a solution of said polyurethane-urea.

Thus, specific compositions and methods of producing a polyurethane-urea elastomer exhibiting ultra-high ultimate elongation have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Having generally described this invention, a further understanding can be obtained by reference to certain specific examples which are provided herein for purposes of illustration only and are not intended to be limiting unless otherwise specified.

### EXAMPLES

### Example 1 (Prepolymer Synthesis)

A prepolymer was synthesized by the reaction between a symmetric diisocyanate with polyether diol with ultralow unsaturation and narrow polydispersity. A pre-dried, jacketed 2,000 ml glass flask was assembled with a six-port glass head equipped with an overhead stirring assembly with a PTFE lined anchor stirrer, thermocouple sensor, nitrogen inlet, liquid dripping funnel, reflux condenser, and 18 mm O.D. column filled with dried 5 Å molecular sieve beads, which was connected to a vacuum system. 1,072 g of Acclaim® 4200 polyether diol (MW 4000, hydroxyl value 28.0 mg KOH/g, OH equivalent weight 2000) and 2.0 g PTFE boiling stones were added to the flask. Nitrogen was bubbled into the polyether diol under slow stirring for 20 minutes. The system was vacuumed down to 1-0.1×10−3 Torr and heated to around 110° C. for 1 hour. Afterward, the vacuum system was removed and the temperature was reduced to 18° C. 50 mL of zirconium (IV) tetra(3-ethylhexanoate) (99 w/w %) was added into the flask. The ratio of NCO/OH was kept at 2.09 and 94.5 g of hexamethylene-1,6-diisocyanate (HDI, Desmodur® H from Bayer, molecular weight of 168, assay [symbol] 99.5%, NCO [symbol] 49.7%, and equivalent weight of 84) was introduced into the addition funnel and added to the stirred mixture drop-wise over a period of one hour under a nitrogen blanket. The temperature of the stirred reaction mixture was kept below 50° C. for 30 minutes, and then slowly heated to 65° C. over the course of 30 minutes. After the addition of HDI, the mixture temperature was heated to 80° C. and kept at 80° C. to 82° C. for an additional half hour. A probe sample was tested using FT-IR, which showed the disappearance of the conformed free OH group stretching at around 3450 cm⁻¹ and appearance of NH group stretching at around 3320 cm⁻¹. The temperature of the system was reduced to room temperature (20° C.), boiling stones were removed, and 0.01 g of double metal cyanide was mixed in to keep the prepolymer stable. A total of 1,165 g of isocyanato terminated prepolymer was obtained. The isocyanato terminated prepolymer had an equivalent weight of 2,168.

### Example 2 (Polyurethane-Urea Elastomer Synthesis)

Polyurethane-urea elastomer was synthesized by chain extending a prepolymer with a bulky diamine. A jacketed 5,000 ml glass flask was pre-dried and mounted under a six-port glass head equipped with an overhead stirrer assembly with a PTFE lined anchor stirrer, thermocouple sensor, nitrogen inlet, liquid dripping funnel, and reflux condenser. 251 g of the isocyanato terminated prepolymer obtained in Example 1 (equivalent weight of 2,168) was added and cooled to 2° C. A stoichiometric amount of a diamine chain extender solution of 6.7183 g of 2-methyl-1,5-diaminopentane [symbol] 98.5%, equivalent weight of 57.5) in 250 mL of dehydrated isopropanol was cooled to −12° C., introduced into the addition funnel, and added drop-wisely into the isocyanato terminated prepolymer. The mixture temperature was kept between 1-5° C. and viscosity was adjusted by the addition of 500 mL of dried tetrahydrofuran (THF). After the addition of 2-methyl-1,5-diaminopentane, an additional 550 mL of dried isopropanol and 1,000 mL of dried THF were added. The reaction mixture was then heated to 10-15° C. for 30 minutes and finally heated to 20° C. A probe sample was tested using FT-IR, which showed the complete disappearance of the conformed free NCO group stretching at around 2270 cm⁻¹. The elastomer solution was cast on rectangular glass trays pretreated with demolding compound and covered with glass sheets. The cast elastomer samples were demolded after being kept under room temperature for two weeks.

### Example 3 (Stress-Strain and Ultimate Elongation Test)

The stress-strain behavior of the cast elastomer films was measured using a Universal Testing System controlled by software. A bench-top die was used to cut dogbone samples with an overall length of ca. 25 mm and grip section width of ca. 10 mm. The reduced section measured 2.91 mm×10 mm (width×gage length). These dogbones were then tested to failure at a crosshead speed of 25 mm/min and their load vs. displacement values recorded. Five samples were measured and their results were averaged to determine modulus, yield strength, and strain-at-break for each of synthesized materials. Maximum elongation was tested to be over 2000%. The selected samples were sent to third party (Applied Technical Services, Inc., 1049 Triad Court, Marietta, Ga. 30062) to test their physical properties and the results are detailed in Table 1.

### Example 4

The prepolymers were synthesized following Example 1, with a variety of diisocyanates, polyether diols, and various ratios of NCO/OH. Polyurethane-urea elastomers were synthesized following Example 2 with various diamine chain extenders. The ultimate elongation at break of the cast elastomer samples were measured. The variation of polyether polyols, diisocyanates, ratios of NCO/OH, and diamines resulted in variations to polyurethane-urea molecular structures, leading to differences in ultimate elongation.

TABLE 1

| Experimental Run | 1 & 2 | 4 | 5 | 6 | 7 | 8 | 9 | 10 | 11 | 12 |
|---|---|---|---|---|---|---|---|---|---|---|
| Polyol A mol. % | | | 50 | 60 | | | 100 | | | |
| Polyol B mol. % | 100 | 100 | 50 | | 100 | 100 | | 100 | 100 | 100 |
| Polyol C mol. % | | | | 40 | | | | | | |
| Av. mol. Wt. | 4000 | 4000 | 3000 | 4400 | 4000 | 4000 | 2000 | 4000 | 4000 | 4000 |
| Diisocyanate | HDI | HDI | HDI | HDI | MDI | IPDI | HDI | HDI | HDI | HDI |
| Diamine | DY | DY | DY | DY | DY | DY | EDA | EDA | IPDA | PDA |
| NCO/OH mol/mol | 2.09 | 1.85 | 2.06 | 2.05 | 2.07 | 2.05 | 2.06 | 2.08 | 2.06 | 2.05 |
| Shore A | 34 | 44 | | | | | | | | |
| Elongation % | >1860* | 68 | 27 | 210 | 563 | 286 | 41 | 444 | 647 | 75 |

Key to Abbreviations Used:
Polyol A: Acclaim ® 2200
Polyol B: Acclaim ® 4200
Polyol C: Acclaim ® 8200
HDI: 1,6-Hexamethylene diisocyanate
MDI: 4,4′-MDI, Bis(4-isocyanatophenyl)methane
IPDI: 3-isocyanto-methyl-3,5,5-trimethyl-cyclohexylisocyanate (isophorone diisocyanate)
EDA Ethylene Diamine
IPDA 3-Amino-methyl-3,5,5-trimethyl-cyclohexylamine (isophorone diamine)
PDA: 1,3-PDA 1,3-Diaminopropane
DY: 2-MPDA 2-Methyl-1,5-diaminopentane
Elongation %: Elongation-at-break, ultimate elongation.
*Samples reached the maximum capacity of the testing machines elongation capacities. Stress and elongation expected be higher than reported (by Applied Technical Services, Inc.)

1. A polyurethane/urea elastomer containing a plurality of urethane/urea hard segments within a polyether diol soft matrix, comprising the reaction product of:
(A) an isocyanato-terminated prepolymer prepared by the reaction of:
(A1) a diisocyanate with a symmetric molecular structure; and
(A2) a polyether diol having a high molecular weight with ultralow unsaturation of monol contents and narrow polydispersity; and
(A3) a homogeneous catalyst for linear polymerization;
wherein the molar ratio of the NCO functional group of said diisocyanate to OH functional group of said polyether polyol is greater than 2.0;
(B) a bulky diamine chain extender having a continuous carbon chain with at least four-carbon atoms between attached amino groups; and
(C) a solvent, to form a solution of said polyurethane/urea.

2. The polyurethane/urea elastomer of claim 1, wherein said diisocyanate with a symmetric molecular structure is selected from the group consisting of hexamethylene diisocyanate (HDI), 1,4-cyclohexyl diisocyanate (CHDI), 4,4′-dicyclohexyl-methylene diisocyanate (H₁₂MDI), 1,4-phenylene diisocyanate (PPDI), 4,4′-dibenzyl diisocyanate (DBDI), 2,6-toluene diisocyanate (2,6-TDI), 1,5-naphthalene diisocyanate (1,5-NDI), and a mixture thereof.

3. The polyurethane/urea elastomer of claim 1, wherein said polyether diol has a molecular weight exceeding 2,000 Da.

4. The polyurethane/urea elastomer of claim 1, wherein said polyether diol has an unsaturation of monol contents of less than 0.007 meg/g.

5. The polyurethane/urea elastomer of claim 1, wherein said polyether diol has a polydispersity of less than 1.20.

6. The polyurethane/urea elastomer of claim 1, wherein said homogeneous catalyst for linear polymerization is selected from the group consisting of aluminum dionate, aluminum octoate, aluminum tris(2,4-pentanedionate), bismuth carboxylate; amine-cuprous chloride, calcium octoate, iron octoate; silver oxide, silver nitrate, silver, nitrite, tin carboxylate, tin octoate, tin II neodecanoate, tin II octoate, tin II oleate, dibutyltin diacetate, dibutyltin dilaurate, dibutyltin diisooctylmaleate, dimethyltin dineodecanoate, dioctyltin dilaurate, bis(2-ethylhexanoate)tin, stannous octoate, bis(2-ethylhexanoate)tin, bis(neodecanoate)tin, di-n-butyl bis(2-ethylhexylmaleate)tin, di-n-butyl bis(2,4-petanedionate)tin, di-n-butylbutoxychlorotin, di-n-butyldiacetoxytin, di-n-butyldilauryltin, di-n-butyldilauryltin, dimethyldineodecanoatetin, dimethylhydroxy(oleate)tin, dioctyldilauryltin, titanium di-n-butoxide, titanium (bis-2,4-pentanedionate), titanium diisopropoxide, titanium 2-ethylhexoxide, titanium (bis-2,4-pentanedionate), titanium diisopropoxide, titanium bis(ethylacetoacetate), titanium 2-ethylhexoxide, titanium trimethylsiloxide, zinc octoate, zirconium dionate, zirconium 2-ethylhexanenoriate, zirconium tetrakis(2,4-pentanedionate) complex, 1,4-diazabicyclo[2,2,2]octane (DABCO), 1,8-diazabicyclo[5,4,0] undec-7-ene (DBU), 1,5,7-triazobicyclo[4,4,0]dec-5-ene (TBD), N-methyl-1,5,7-triazobicyclododecene (MTBD), diethanolamine, 3-dimethylamino-N,N-dimethylpropioamide, bis[2-dimethylaminoethyl]ether, N-[2-(dimethylamino)ethyl]-N-methylethanolamine, 2[2-dimethylaminoethyoxyl]ethanol, 3-dimethylamino-N,N-dimethylpropioamide, N,N-dimethylcyclohexylamine, dimethylethanolamine, N-ethylmorpholine, N,N,N′,N′,N′-pentamethyldiethylenetriamine, tertiary phosphines, tetrachlorocuprate having anilinium, p-methoxyanililinum, p-hethylanilium and pyridium cations, triethylamine, triethylenediamine, and a mixture thereof.

7. The polyurethane/urea elastomer of claim 1, wherein said bulky diamine chain extender is selected from the group consisting of 1,4-diaminobutane, 1,5-dimanopentane, 1,6-diaminohexane, 2-methyl-1,5-diaminopentane, 3-methyl-1,5-diaminopentane, 2-methyl-1,6-diaminohexane, 3-methyl-1,6-diaminohexane, 1,8-diaminooctane, 1,10-diaminodecane, 1,12-diaminododecane, trans-1,4-cyclohexyl diamine (t-CHDA), trans-1,8-diamino-p-menthane, trans,trans-4,4′-dicyclohexyl-methylene diamine (t,t-H₁₂MDA), 2,2-dimethyl-1,3-propanediamine (neopentadiamine), 4,4-diamino-dibenzyl (DAB), p-phenyl diamine (1,4-phenylenediamine) (PPDA), 1,5-naphthalene diamine (1,5-NDA), 2,6-toluene diamine (2,6-TDA), p-xylylenediamine, 1-isopropylamino-3-methylene isopropylamino-3,5,5-trimethyl-cyclohexane (Jefflink 754), 4,4′-dibutylaminodicyclohexyl methane (Clearlink 1000), polyaspartic ester diamine, aldimine, polyalkyl diamine, polyoxypropylene diamine, (polyethylene glycol) diamine, 2,5-bis(4-amino-phenylene)-1,3,4-oxadiazole (DAPO), 2,6-diamino-pyridine (DAPY), and a mixture thereof.

8. The polyurethane/urea elastomer of claim 1, wherein said solvent is selected from the group consisting of acetone, acetonitrile, acetophenone, amyl acetate, benzyl benzoate, butanol, 2-butanol, butanone, butyl acetate, sec-butyl acetate, tert-butyl acetate, gama-butylolactone, n-butyl propionate, para-chlorobenzotrifluoride, chloroform, cyclobutanone, cyclohexane, cyclohexanone, cyclopentanone, dichloromethane, diethyl carbonate, diethyl ketone, diisobutyl ketone, N,N-dimethylacetamide, dimethyl carbonate, N,N-dimethyl ethylene urea, N,N-dimethylformamide, dimethyl sulfoxide, dioctyl terephthalate, 1,4-dioxane, ethanol, 2-ethoxyethyl ether, ethyl acetate, ethyl acetoacetate, ethyl butyrate, ethyl lactate, ethylene carbonate, bis(2-ethylhexyl) adipate, ethyl isopropyl ketone, hexyl acetate, isoamyl acetate, isobutanol, isobutyl acetate, isobutyl isobutyrate, isopropanol, isopropyl acetate, isophorone, mesityl oxide, methanol, methyl acetate, methyl amyl acetate, methyl butyl ketone, methyl chloroform, methylene chloride, methyl ethyl ketone, methyl isoamyl ketone, methyl isobutyl ketone, methyl isopropyl ketone, methyl propyl ketone, N-methyl-2-pyrrolidinone, octyl acetate, 3-pentanone, n-pentyl propionate, perchloroethylene, propanol, 2-propanol, beta-propyolactone, propyl acetate, tetrahydrofuran, tetramethylene sulfone, toluene, triacetin, delta-valerolactone, xylene, and a mixture thereof.

9. A polyurethane/urea elastomer containing a plurality of urethane/urea hard segments within a polyether-co-polycarbonate soft matrix, comprising the reaction product of:
(A) an isocyanato-terminated prepolymer prepared by the reaction of:
(A1) a diisocyanate with a symmetric molecular structure; and
(A2) a polyether-co-polycarbonate diol having a high molecular weight with ultralow unsaturation of monol contents and narrow polydispersity; and
(A3) a homogeneous catalyst for linear step polymerization;
wherein the molar ratio of the NCO functional group of said diisocyanate to OH functional group of said polyether-co-polycarbonate diol is greater than 2.0;
(B) a bulky diamine chain extender having a continuous carbon chain with at least four-carbon atoms between attached amino groups; and
(C) a solvent, to form a solution of said polyurethane/urea.

10. The polyurethane/urea elastomer of claim 9, wherein said diisocyanate with a symmetric molecular structure is selected from the group consisting of hexamethylene diisocyanate (HDI), 1,4-cyclohexyl diisocyanate (CHDI), 4,4′-dicyclohexyl-methylene diisocyanate (H₁₂MDI), 1,4-phenylene diisocyanate (PPDI), 4,4′-dibenzyl diisocyanate (DBDI), 2,6-toluene diisocyanate (2,6-TDI), 1,5-naphthalene diisocyanate (1,5-NDI), and a mixture thereof.

11. The polyurethane/urea elastomer of claim 9, wherein said polyether-co-polycarbonate diol has a molecular weight exceeding 2,000 Da.

12. The polyurethane/urea elastomer of claim 9, wherein said polyether-co-polycarbonate diol has an unsaturation of monol contents of less than 0.007 meg/g.

13. The polyurethane/urea elastomer of claim 9, wherein said polyether-co-polycarbonate diol has a polydispersity less than 1.20.

14. The polyurethane/urea elastomer of claim 9, wherein said homogeneous catalyst for linear polymerization is selected from the group consisting of aluminum dionate, aluminum octoate, aluminum tris(2,4-pentanedionate), bismuth carboxylate; amine-cuprous chloride, calcium octoate, iron octoate; silver oxide, silver nitrate, silver, nitrite, tin carboxylate, tin octoate, tin II neodecanoate, tin II octoate, tin II oleate, dibutyltin diacetate, dibutyltin dilaurate, dibutyltin diisooctylmaleate, dimethyltin dineodecanoate, dioctyltin dilaurate, bis(2-ethylhexanoate)tin, stannous octoate, bis(2-ethylhexanoate)tin, bis(neodecanoate)tin, di-n-butyl bis(2-ethylhexylmaleate)tin, di-n-butyl bis(2,4-petanedionate)tin, di-n-butylbutoxychlorotin, di-n-butyldiacetoxytin, di-n-butyldilauryltin, di-n-butyldilauryltin, dimethyldineodecanoatetin, dimethylhydroxy(oleate)tin, dioctyldilauryltin, titanium di-n-butoxide, titanium (bis-2,4-pentanedionate), titanium diisopropoxide, titanium 2-ethylhexoxide, titanium (bis-2,4-pentanedionate), titanium diisopropoxide, titanium bis(ethylacetoacetate), titanium 2-ethylhexoxide, titanium trimethylsiloxide, zinc octoate, zirconium dionate, zirconium 2-ethylhexanenoriate, zirconium tetrakis(2,4-pentanedionate) complex, 1,4-diazabicyclo[2,2,2]octane (DABCO), 1,8-diazabicyclo[5,4,0] undec-7-ene (DBU), 1,5,7-triazobicyclo[4,4,0]dec-5-ene (TBD), N-methyl-1,5,7-triazobicyclododecene (MTBD), diethanolamine, 3-dimethylamino-N,N-dimethylpropioamide, bis[2-dimethylaminoethyl]ether, N-[2-(dimethylamino)ethyl]-N-methylethanolamine, 2[2-dimethylaminoethyoxyl]ethanol, 3-dimethylamino-N,N-dimethylpropioamide, N,N-dimethylcyclohexylamine, dimethylethanolamine, N-ethylmorpholine, N,N,N′,N′,N′-pentamethyldiethylenetriamine, tertiary phosphines, tetrachlorocuprate having anilinium, p-methoxyanililinum, p-hethylanilium and pyridium cations, triethylamine, triethylenediamine, and a mixture thereof.

15. The polyurethane/urea elastomer of claim 9, wherein said bulky diamine chain extender is selected from the group consisting of 1,4-diaminobutane, 1,5-dimanopentane, 1,6-diaminohexane, 2-methyl-1,5-diaminopentane, 3-methyl-1,5-diaminopentane, 2-methyl-1,6-diaminohexane, 3-methyl-1,6-diaminohexane, 1,8-diaminooctane, 1,10-diaminodecane, 1,12-diaminododecane, trans-1,4-cyclohexyl diamine (t-CHDA), trans-1,8-diamino-p-menthane, trans,trans-4,4′-dicyclohexyl-methylene diamine (t,t-H₁₂MDA), 2,2-dimethyl-1,3-propanediamine (neopentadiamine), 4,4-diamino-dibenzyl (DAB), p-phenyl diamine (1,4-phenylenediamine) (PPDA), 1,5-naphthalene diamine (1,5-NDA), 2,6-toluene diamine (2,6-TDA), p-xylylenediamine, 1-isopropylamino-3-methylene isopropylamino-3,5,5-trimethyl-cyclohexane (Jefflink 754), 4,4′-dibutylaminodicyclohexyl methane (Clearlink 1000), polyaspartic ester diamine, aldimine, polyalkyl diamine, polyoxypropylene diamine, (polyethylene glycol) diamine, 2,5-bis(4-amino-phenylene)-1,3,4-oxadiazole (DAPO), 2,6-diamino-pyridine (DAPY), and a mixture thereof.

16. The polyurethane/urea elastomer of claim 9, wherein said solvent is selected from the group consisting of acetone, acetonitrile, acetophenone, amyl acetate, benzyl benzoate, butanol, 2-butanol, butanone, butyl acetate, sec-butyl acetate, tert-butyl acetate, gamma-butylolactone, n-butyl propionate, para-chlorobenzotrifluoride, chloroform, cyclobutanone, cyclohexane, cyclohexanone, cyclopentanone, dichloromethane, diethyl carbonate, diethyl ketone, diisobutyl ketone, N,N-dimethylacetamide, dimethyl carbonate, N,N-dimethyl ethylene urea, N,N-dimethylformamide, dimethyl sulfoxide, dioctyl terephthalate, 1,4-dioxane, ethanol, 2-ethoxyethyl ether, ethyl acetate, ethyl acetoacetate, ethyl butyrate, ethyl lactate, ethylene carbonate, bis(2-ethylhexyl) adipate, ethyl isopropyl ketone, hexyl acetate, isoamyl acetate, isobutanol, isobutyl acetate, isobutyl isobutyrate, isopropanol, isopropyl acetate, isophorone, mesityl oxide, methanol, methyl acetate, methyl amyl acetate, methyl butyl ketone, methyl chloroform, methylene chloride, methyl ethyl ketone, methyl isoamyl ketone, methyl isobutyl ketone, methyl isopropyl ketone, methyl propyl ketone, N-methyl-2-pyrrolidinone, octyl acetate, 3-pentanone, n-pentyl propionate, perchloroethylene, propanol, 2-propanol, beta-propyolactone, propyl acetate, tetrahydrofuran, tetramethylene sulfone, toluene, triacetin, delta-valerolactone, xylene, and a mixture thereof.
